(19) United States
(12) Patent Application Publication
(10) Pub. No.: US 2010/0312307 A1
(43) Pub. Date: Dec. 9, 2010

(54) ELECTROTHERAPY APPARATUS

(76) Inventor: Conor Minogue, Kinvara (IE)

Correspondence Address: WINSTEAD PC, P.O. BOX 50784, DALLAS, TX, 75201, US

(21) Appl. No.: 12/863,511

(22) PCT Filed: Jan. 5, 2009
(86) PCT No.: PCT/IB2009/050015

§ 371 Date: Aug. 16, 2010

(30) Foreign Application Priority Data
Jan. 30, 2008 (GB) 0801611.5

(51) Int. Cl. A61N 1/36

(52) U.S. Cl. 607/48

(57) ABSTRACT

The invention relates to an electrotherapy apparatus facilitating accurate and repeatable application to a neuromuscular area to be treated by a patient. The apparatus comprises a flexible garment (12) adapted to be worn by the patient. Stimulation electrodes (10) are attachable to the body-facing surface of the garment (12) on at least a predetermined portion(s) thereof. An array of spaced through passages (14) are provided in two dimensions throughout the surface area of at least the predetermined portion(s) of the garment (12) to allow passage of a connection means, such as a lead wire (L) or a stud fastener, connecting the electrode (10) to an external stimulation apparatus. The invention also discloses a method of preparing the electrotherapy apparatus for subsequent use by the patient.

The present invention relates to an electrotherapy apparatus and particularly, but not exclusively, to an electrotherapy apparatus using lead wire electrodes and to a method for preparing same for accurate and repeatable application by a patient to a neuromuscular area to be treated.

In electrotherapy there is frequently a requirement for careful selection and positioning of electrodes on the body by a professional therapist in order to achieve an optimal therapeutic effect. Firstly, the therapist selects the type, size and shape of electrodes to fix to a body part. Sometimes the electrodes to be applied have different shapes or sizes. Frequently the therapist will have cause to reposition one or more of the electrodes to improve the effectiveness of the therapy. Having thus identified the size, shape, number and location of electrodes on a body part it is often required that the patient continue the treatment at home for a number of weeks, without specialist assistance. There is a risk that the patient will not reproduce the exact electrode configuration and there is therefore a need for an easy to use applicator that accurately reproduces the optimised electrode configuration determined by the therapist.

According to a first aspect of the present invention, there is provided an electrotherapy apparatus for accurate and repeatable placement of stimulation electrodes to a neuromuscular area to be treated, the apparatus comprising:

(i) a flexible garment for application to a neuromuscular area to be treated, the garment being provided with a plurality of spaced through-passages each extending between its body-facing surface and an opposing surface;
(ii) means for securing the garment against a neuromuscular area to be treated;
(iii) at least one stimulation electrode provided with connection means for connection to an external stimulation apparatus, each stimulation electrode being attachable to the body-facing surface of the garment; and
(iv) cooperating attachment means provided on each stimulation electrode and on at least a predetermined portion(s) of the surface area of the body-facing surface of the flexible garment respectively for attaching each stimulation electrode to the garment; the cooperating means on the body-facing surface of the garment being provided over the entire surface area of the predetermined portion(s) to allow unfettered attachment of each stimulation electrode at any position thereon;
characterised in that the spaced through-passages are arranged in an array such that the passages are spaced in two dimensions throughout the surface area of at least said predetermined portion(s) of the garment to facilitate passage of said connection means from a proximately positioned stimulation electrode, for connection to an external stimulation apparatus, irrespective of the location of the stimulation electrode on the body-facing surface of the garment.

Preferably, the connection means is a lead wire.

Lead wires are the most common type of electrode used in clinical practice and are compatible with the majority of stimulation apparatus.

Alternatively, the connection means is a stud fastener.

Preferably, the flexible garment is resiliently deformable in at least the predetermined portion(s) to facilitate the passage of the connection means.

Preferably, the passages neighbouring passages are equi-spaced in two dimensions.

Preferably, the maximum spacing between neighbouring passages is six centimetres.

The length of a typical electrode's lead wire is approximately seven centimetres and therefore such a maximum spacing of passages ensures that any point on an electrode attached to any point of the predetermined portion of the garment will be within a maximum of three centimetres from a passage. This ensures that a sufficient length of the electrode's lead wire will extend through the passage for ease of connection to a lead wire of an external stimulation device. When stud fasteners are used, the maximum spacing may be reduced to avoid over-stretching of the flexible garment when locating the nearest passage.

Preferably, the passages are circular in cross section.

The passages may be formed by punching through the garment material and should be able to accommodate both an electrode's lead wire and its connecting terminal. To avoid the need for larger diameter passages, the material may optionally be resilient, at least in the region(s) of each passage, to facilitate temporary enlargement of each passage. In this way, the lead wire and its connecting terminal can be more easily passed through a manually enlarged passage which returns to its smaller size to grip the lead wire upon removal of the manual stretching force. Ideally, the diameter of each passage should be 2 mm. Resilience is particularly beneficial when stud fasteners are used.

Preferably, each electrode is removably attachable to the garment.

Such an arrangement facilitates repositioning of the electrodes such that their placement can be optimised, or be changed to target a different neuromuscular area.

Preferably, the cooperating attachment means is provided in the form of hook and loop fasteners.

Preferably, the hooks are provided on the electrodes and the loops are provided on the garment.

Preferably, the means for securing the garment against a neuromuscular area to be treated may be selected from the group comprising hook and loop fasteners, zip fasteners, buckles and buttons.

Preferably, the predetermined portion(s) of the surface area of the body-facing surface of the flexible garment are dependent on the neuromuscular area to be treated and correspond with the areas where electrodes are most likely to be required.

It is not necessary for the entire body-facing surface of the garment to be provided with, for example, loop material. This is particularly true when a garment for a particular part of the body leads to a likelihood that electrodes will only be required in certain predefined areas.

Preferably, starting from their body-facing surface, each electrode comprises an adhesive conductive layer; a conductive current dispersion layer; an insulating backing layer; and a layer of hook or loop material.

Preferably, each electrode is additionally provided with a removable liner layer for covering the adhesive conductive layer before attachment of the electrode to the body, or after its removal.

Preferably, the hook and loop fasteners are adapted to have a relatively higher peel strength than that between the adhesive conductive layer and the skin on the neuromuscular area to be treated.

This arrangement serves to ensure that each electrode remains attached to the body-facing surface of the garment upon its removal from the neuromuscular area to be treated.

Optionally, lead wire retaining means are provided on said opposing surface of the garment for releasably retaining the lead wires in position.

Optionally, the garment is configured for securing against the knee, shoulder, ankle, back or abdomen.

According to a second aspect of the present invention, there is provided a method for preparing an electrotherapy apparatus for accurate and repeatable application by a patient to a neuromuscular area to be treated, the method comprising the steps of:

(i) selecting at least one stimulation electrode provided with connection means for connection to an external stimulation apparatus, and being suitable for application to a neuromuscular area to be treated;
(ii) removably attaching a relatively low peel strength electrically conductive layer of each electrode to the neuromuscular area to be treated;
(iii) connecting an external stimulation apparatus to each electrode;
(iv) electrically stimulating the neuromuscular area to be treated;
(v) assessing the response of the neuromuscular area to be treated to the electrical stimulation;
(vi) optionally removing and reattaching one or more of the electrodes and repeating steps (iv) and (v) until a desired response is obtained;
(vii) disconnecting the external stimulation apparatus from each electrode;
(viii) applying a flexible garment provided with an array of spaced passages extending between its body-facing surface and an opposing surface onto the neuromuscular area to be treated and, in doing so, removably coupling cooperating attachment means having a relatively high peel strength on each electrode and on at least a predetermined portion(s) of the surface area of the body-facing surface of the flexible garment respectively;
(ix) removing the flexible garment from the neuromuscular area to be treated whilst retaining each electrode thereon by virtue of the differential peel strengths;
(x) feeding the connection means from each electrode through the nearest passage in the garment in readiness for subsequent connection to an external stimulation apparatus.

Preferably, the step of applying a flexible garment onto the neuromuscular area to be treated is assisted by identifying one or more reference points on the garment and aligning the or each reference point with one or more corresponding anatomical landmarks on the neuromuscular area to be treated.

For example, for the upper leg, suitable anatomical landmarks could be the midline of the leg and the patella.

Preferably, the step of applying a flexible garment onto the neuromuscular area to be treated involves securing the garment against a body part using one or more of hook and loop fasteners, zip fasteners, buckles and buttons.

Preferably, the step of applying a flexible garment onto the neuromuscular area to be treated involves securing the garment against the knee, shoulder, ankle, back or abdomen.

Preferably, steps (i) to (x) are implemented by a therapist in readiness for subsequent connection to an external stimulation apparatus by a patient.

Embodiments of the present invention will now be described by way of example only with reference to the accompanying drawings, in which:

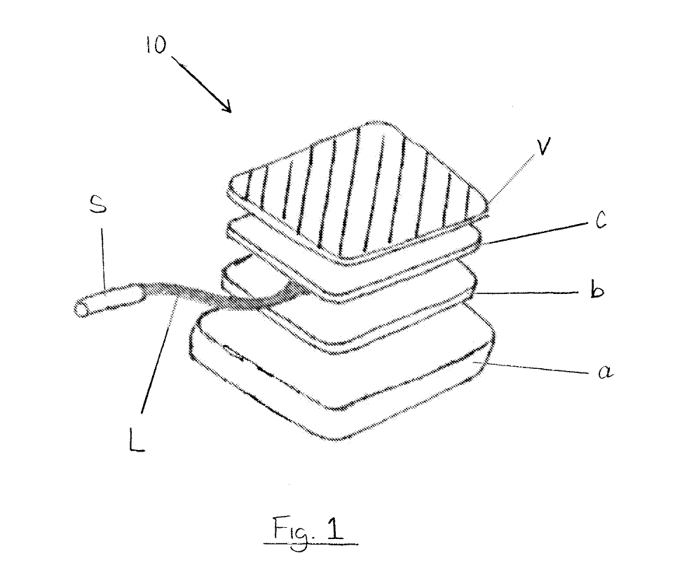

FIG. 1 is a schematic partially exploded view of a preferred lead wire type electrode having opposing surfaces with differential peel strengths;

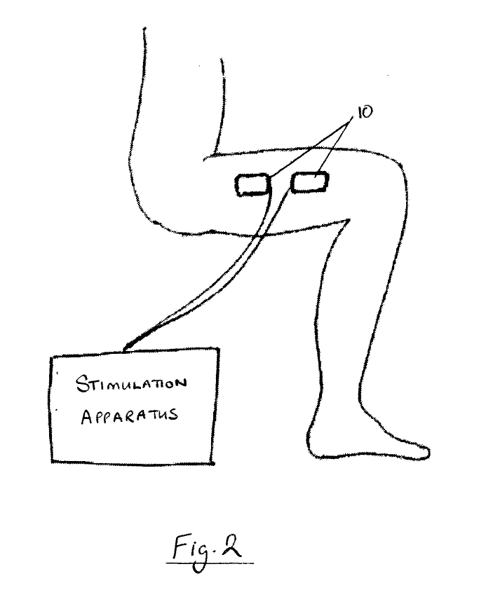

FIG. 2 is a schematic view of the placement of electrodes on a patient's leg (by a therapist) wherein each electrode is connected to external stimulation apparatus;

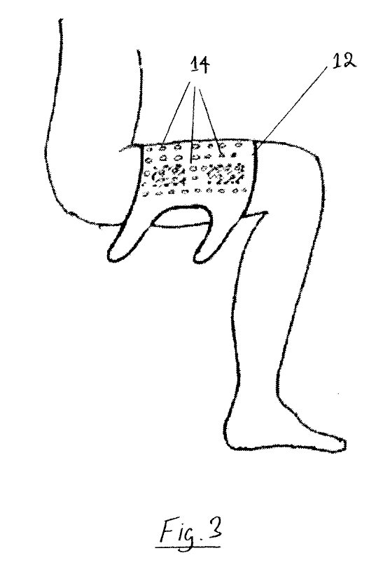

FIG. 3 is a schematic view of a flexible garment wrapped around the leg (by a therapist) over the electrodes;

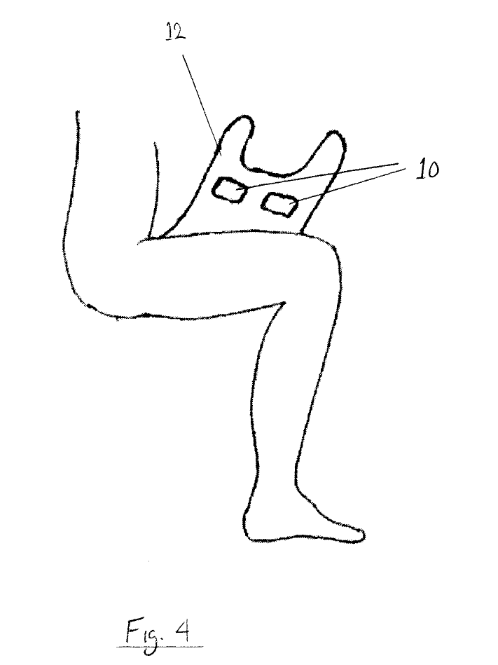

FIG. 4 is a schematic view of the flexible garment being unwrapped from the leg (by a therapist) with the electrodes remaining attached to its body-facing surface;

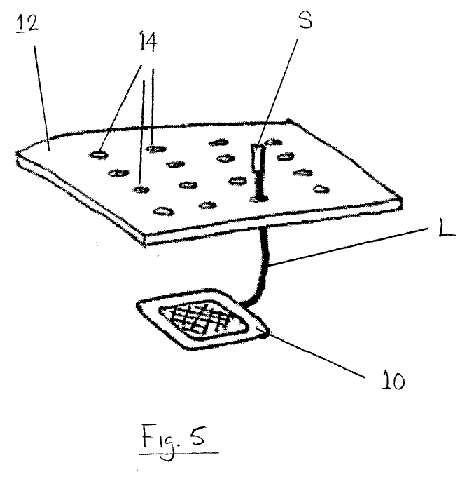

FIG. 5 is a schematic view of an electrode's lead wires being fed through one of an array of passages provided on the flexible garment (by a therapist); and

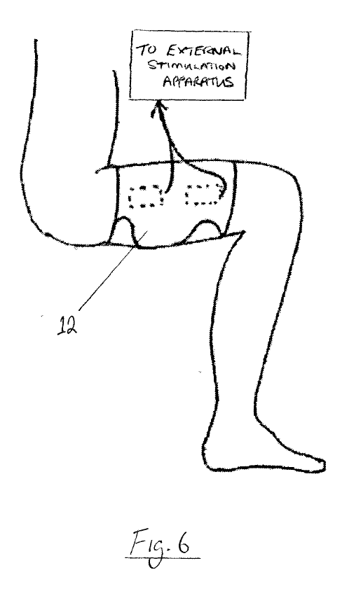

FIG. 6 is a schematic view showing the re-application of the garment to the leg (by the patient) to accurately reproduce the optimised positioning of the electrodes as determined by a therapist.

Lead wire stimulation electrodes (10) forming part of the apparatus of the present invention are shown in FIG. 1. The electrodes (10) are based on the conventional design, comprising a layer of conductive adhesive hydrogel (a); a conductive dispersive layer (b), a short flexible lead wire (L), and an insulated backing layer (c). The conductive adhesive hydrogel layer (a) is suitable for removable attachment to the skin as indicated in FIG. 2 and described in greater detail below. The short flexible lead wire (L) has a typical length of about seven centimetres and emerges from between the conductive dispersive layer (b) and the insulated backing layer (c). The lead wire (L) terminates in a 2 mm female socket connector (S). This connector (S) is intended to receive a corresponding 2 mm moulded terminal pin found on a wide range of commercial stimulation device lead wires. Electrodes of the lead wire type are more commonly used in clinical practice and, unlike stud-type electrodes, are compatible with most electrical stimulation apparatus.

The electrodes (10) of the present invention differ from conventional electrodes in that they are provided with a layer of fastening material such as Velcro® hook material (V) on their outer facing surface. The Velcro® hook material (V) facilitates repeated attachment and removal of the electrodes (10) to the body facing surface of a garment as is described in detail below.

FIGS. 3, 4 and 6, show a flexible garment (12) that includes one or more markers (not shown) for alignment with one or more anatomical features of the body such that its location on the body can be easily reproduced. For example, the flexible garment (12) is shown in the figures wrapped around the upper leg and secured in position by means of loop fasteners provided on the inner body-facing surfaces of its opposing distal ends and corresponding loop fasteners on at least part of its opposite surface. Of course, alternative fastening means are possible such as zip fasteners, buckles and buttons etc. Such a garment (12) could be provided with markers (not shown) to locate it with respect to the midline of the leg and the patella. An example of such an arrangement is disclosed in the applicant's co-pending application: WO2004/098703A.

The flexible garment (12) is provided with a two-dimensional array of spaced through-passages (14) of a circular cross-section extending between its body-facing surface and its opposing surface. The through passages (14) are provided at least in those predetermined areas were electrodes (10) are likely to be required on the body (i.e. depending upon the particular neuromuscular area to be treated). The passages may be arranged in a matrix type pattern of regular rows and columns where neighbouring passages are equi-spaced with a maximum spacing of approximately 5-7 cm, but most preferably a maximum of 6 cm. The body-facing surface of the flexible garment (12) is provided with a fastening material, such as Velcro® loop material, which will not attach to the skin but which will attach to a corresponding Velcro® hook material provided on the stimulation electrodes (10) as described above. It will appreciated that any suitable alternative cooperating attachment means to Velcro® hook and loop material may be provided on the stimulation electrodes (10) and predetermined portion(s) of the flexible garment (12) respectively. The fastening material on the body-facing surface of the flexible garment (12) is also provided at least in those predetermined areas were electrodes (10) are likely to be required on the body. Importantly, the peel strength of the hook and loop fasteners is greater than the peel strength of the each electrode's conductive adhesive hydrogel layer (a).

The procedure for using the invention is as follows. Firstly the therapist selects the number and type of lead wire stimulation electrodes (10) which are thought to be appropriate to for the intended treatment location. She removes any protective liners on the skin-facing adhesive hydrogel layer (a) and attaches the electrodes (10) to the skin in the normal way. She connects the lead wires of an external electrotherapy stimulation device to the lead wires (L) of the stimulation electrodes (10) as shown in FIG. 2.

She adjusts the external electrotherapy stimulation device to achieve the desired response and, if necessary, she may adjust the position of, or replace one or more of the electrodes (10) by a different type. When she is satisfied with the setup she turns off the external electrotherapy stimulation device and detaches the stimulator lead wires from the lead wires (L) of each electrode (10).

She then takes the flexible garment (12) which is intended for the body part in question and identifies the reference markings thereon which are intended to align with corresponding anatomical landmarks on the body. She wraps the garment (12) around the body part and attaches it firmly against the body part to be treated, i.e. by pressing firmly against the body part such that the respective Velcro® hook and loop material on the electrodes (10) and the predetermined portion(s) of the body-facing surface of the garment (12) are properly attached as shown in FIG. 3.

She then removes the garment (12) from the body part and, in doing so, the electrodes (10) detach from the skin and remain in position on the body-facing surface of the garment as shown in FIG. 4. One the garment is removed from the body, the lead wires (L) from each electrode are fed through the nearest available passage (14) in the garment (12) without disturbing the position of the electrodes (10) on the inner surface of the garment as shown in FIG. 5. Finally, she replaces any electrode liners (not shown) to protect the adhesive hydrogel layer (a) on the electrodes (10). The apparatus is then ready for reliable and repeatable positioning on the body and subsequent connection to an external stimulation apparatus by a patient without any supervision from a therapist.

For home use, the patient is instructed to remove the liners from the electrodes (10), wrap and close the garment (12) around the body part so that the reference markings on the garment (12) align with appropriate anatomical landmarks on the body. The electrode positions selected by the therapist will now be correct and the patient can adjust the external stimulation apparatus to receive the proper treatment as shown in FIG. 6.

Modifications and improvements may be made to the foregoing apparatus and method without departing from the scope of the present invention. For example, any suitable retaining means may be attached to the exterior surface of the garment (12) retain/align lead wires (L) in position against the garment (12). Whilst the figures depict the invention as applied to the upper leg or knee, it is equally applicable to other body parts, for example, the shoulder, ankle, back and abdomen.

Whilst the preferred type of stimulation electrode would be a lead wire electrode, stud fastener type electrodes could alternatively or additionally be employed. In such cases a female snap fastener on the external stimulation device would be attachable to a corresponding male stud on the electrode. The invention could easily be adapted to work with such a stud fastener electrode by pressing the stud portion through an appropriate passage in the flexible garment for subsequent attachment to the external stimulation device.

1. An electrotherapy apparatus for accurate and repeatable placement of stimulation electrodes to a neuromuscular area to be treated, the apparatus comprising:
(i) a flexible garment for application to a neuromuscular area to be treated, the flexible garment being provided with a plurality of spaced through-passages each extending between its body-facing surface and an opposing surface;
(ii) means for securing the flexible garment against a neuromuscular area to be treated;
(iii) at least one stimulation electrode provided with connection means for connection to an external stimulation apparatus, each stimulation electrode being attachable to the body-facing surface of the flexible garment; and
(iv) cooperating attachment means provided on each stimulation electrode and on at least a predetermined portion(s) of the surface area of the body-facing surface of the flexible garment respectively for attaching each stimulation electrode to the flexible garment;
wherein the cooperating attachment means on the body-facing surface of the flexible garment being provided over the entire surface area of the predetermined portion(s) to allow unfettered attachment of each stimulation electrode at any position thereon; and
wherein the spaced through-passages are arranged in an array such that the passages are spaced in two dimensions throughout the surface area of at least said predetermined portion(s) of the flexible garment to facilitate passage of said connection means from a proximately positioned stimulation electrode, for connection to an external stimulation apparatus, irrespective of the location of the stimulation electrode on the body-facing surface of the flexible garment.

2. An electrotherapy apparatus as claimed in claim 1, wherein the connection means is a lead wire.

3. An electrotherapy apparatus as claimed in claim 1, wherein the connection means is a stud fastener.

4. An electrotherapy apparatus as claimed in claim 1, wherein the flexible garment is resiliently deformable in at least the predetermined portion(s) to facilitate the passage of the connection means.

5. An electrotherapy apparatus as claimed in claim 1, wherein neighbouring passages are equi-spaced in two dimensions.

6. An electrotherapy apparatus as claimed in claim 5, wherein maximum spacing between the neighbouring passages is six centimetres.

7. An electrotherapy apparatus as claimed in claim 1, wherein the passages are circular in cross section.

8. An electrotherapy apparatus as claimed in claim 1, wherein each electrode is removably attachable to the flexible garment.

9. An electrotherapy apparatus as claimed in claim 8, wherein the cooperating attachment means is provided in the form of hook and loop fasteners.

10. An electrotherapy apparatus as claimed in claim 9, wherein the hooks are provided on the electrodes and the loops are provided on the flexible garment.

11. An electrotherapy apparatus as claimed in claim 1, wherein the means for securing the flexible garment against a neuromuscular area to be treated may be selected from the group consisting of hook and loop fasteners, zip fasteners, buckles and buttons.

12. An electrotherapy apparatus as claimed in claim 1, wherein the predetermined portion(s) of the surface area of the body-facing surface of the flexible garment are dependent on the neuromuscular area to be treated and correspond with the areas where electrodes are most likely to be required.

13. An electrotherapy apparatus as claimed in claim 1, wherein, starting from their body-facing surface, each electrode comprises:
an adhesive conductive layer;
a conductive current dispersion layer;
an insulating backing layer; and
a layer of hook or loop material.

14. An electrotherapy apparatus as claimed in claim 13, wherein each said electrode is additionally provided with a removable liner layer for covering the adhesive conductive layer before attachment of the electrode to the body, or after its removal.

15. An electrotherapy apparatus as claimed in claim 11, wherein the hook and loop fasteners are adapted to have a relatively higher peel strength than that of the adhesive conductive layer on the neuromuscular area to be treated.

16. An electrotherapy apparatus as claimed in claim 2, wherein lead wire retaining means are provided on said opposing surface of the flexible garment for releasably retaining the lead wires in position.

17. An electrotherapy apparatus as claimed in claim 1, wherein the flexible garment is configured for securing against the knee, shoulder, ankle, back or abdomen.

18. A method for preparing an electrotherapy apparatus for accurate and repeatable application by a patient to a neuromuscular area to be treated, the method comprising the steps of:
(i) selecting at least one stimulation electrode provided with connection means for connection to an external stimulation apparatus, and being suitable for application to a neuromuscular area to be treated;
(ii) removably attaching a relatively low peel strength electrically conductive layer of each electrode to the neuromuscular area to be treated;
(iii) connecting an external stimulation apparatus to each electrode;
(iv) electrically stimulating the neuromuscular area to be treated;
(v) assessing the response of the neuromuscular area to be treated to the electrical stimulation;
(vi) optionally removing and reattaching one or more of the electrodes and repeating steps (iv) and (v) until a desired response is obtained;
(vii) disconnecting the external stimulation apparatus from each electrode;
(viii) applying a flexible garment provided with an array of spaced passages extending between its body-facing surface and an opposing surface onto the neuromuscular area to be treated and, in doing so, removably coupling cooperating attachment means having a relatively high peel strength on each electrode and on at least a predetermined portion(s) of the surface area of the body-facing surface of the flexible garment respectively;
(ix) removing the flexible garment from the neuromuscular area to be treated whilst retaining each electrode thereon by virtue of the differential peel strengths; and
(x) feeding the connection means from each electrode through the nearest passage in the flexible garment in readiness for subsequent connection to an external stimulation apparatus.

19. A method for preparing an electrotherapy apparatus as claimed in claim 18, wherein the step of applying the flexible garment onto the neuromuscular area to be treated is assisted by identifying one or more reference points on the flexible garment and aligning the or each reference point with one or more corresponding anatomical landmarks on the neuromuscular area to be treated.

20. A method for preparing an electrotherapy apparatus as claimed in claim 18, wherein the step of applying the flexible garment onto the neuromuscular area to be treated involves securing the flexible garment against a body part using one or more of hook and loop fasteners, zip fasteners, buckles and buttons.

21. A method for preparing an electrotherapy apparatus as claimed in claim 18, wherein the step of applying the flexible garment onto the neuromuscular area to be treated involves securing the flexible garment against the knee, shoulder, ankle, back or abdomen.

22. A method for preparing an electrotherapy apparatus as claimed in claim 18, wherein steps (i) to (x) are implemented by a therapist in readiness for subsequent connection to an external stimulation apparatus by the patient.
